(19) United States
(12) Patent Application Publication
(10) Pub. No.: US 2025/0057535 A1
(43) Pub. Date: Feb. 20, 2025

(54) Medical Instrument for Avoiding Intraoperative Bleeding

(71) Applicants: Frank Schure, Biesenthal (DE); Viola Schure, Biesenthal (DE)

(72) Inventors: Frank Schure, Biesenthal (DE); Viola Schure, Biesenthal (DE)

(21) Appl. No.: 18/722,798

(22) PCT Filed: Dec. 21, 2022
(86) PCT No.: PCT/EP2022/087304

§ 371 Date: Jun. 21, 2024

(30) Foreign Application Priority Data
Dec. 22, 2021 (DE) 10 2021 134 424.7

(51) Int. Cl. A61B 17/12; A61B 17/00; A61B 17/42

(52) U.S. Cl. CPC A61B 17/12013; A61B 17/42; A61B 2017/00367; A61B 2017/12004

(57) ABSTRACT

A medical instrument (1) for reducing the blood flow through a blood vessel of a human or animal is described, having a band element (2), which is fastened to a handling unit (4) and which has a movable band portion (5), the movement of which makes it possible to change the size and/or shape of a surface area (9), which is enclosed by a loop (6) formed by the band element (2).

The technical solution described is characterized in that at least one fixing means (10) is provided for at least temporarily locking the movable band portion (5), and in that, by actuation of the actuation element (8), the movable band portion (5) can be transferred in a targeted manner from the locked position to a position for increasing or decreasing the surface area (9) enclosed by the loop (6).

### CROSS REFERENCE TO RELATED APPLICATIONS

This application is the U.S. national stage of International Application No. PCT/EP2022/087304, filed on 2022 Dec. 21. The international application claims the priority of DE 102021134424.7 filed on 2021 Dec. 22; all applications are incorporated by reference herein in their entirety.

### BACKGROUND

The invention relates to a medical instrument for avoiding intraoperative bleeding, using which the blood flow from an injured blood vessel of a human or animal can be reduced or stopped. Likewise, this instrument can also be applied in other areas, such as in emergency and/or military medicine for temporary ligation of extremities or organs, for partial removals, such as bowel resections, and/or for uterine removals or partial removals. In addition, such an instrument can be used in plastic surgery for tensioning tissue.

The medical instrument has a relatively rigid, but well bendable band element, which is fastened to a handling unit with a joining region and which has a band portion movable relative to the joining region. The movable band portion, forming a loop, is guided in an adjusting unit, at least indirectly fastened to the handling unit, with an actuation element in such a way that, due to an actuation of the actuation element, the band portion can be moved relative to the joining region and thus a size and/or shape of a surface area enclosed by the loop can be changed.

From the state of the art, different solutions are known to reduce or completely prevent the blood flow from an injured blood vessel of a human or animal after an accident or during surgery. Here, the affected blood vessels are usually squeezed or tied off in order to close the existing opening of the blood vessel.

In this context, a device is known from DE 10 2012 013 153 A1, that enables the ligation of a blood vessel in an extremity. The instrument has a belt to be placed around the extremity in the manner of a sling, a fastener to hold the belt in a tensioned state as required, as well as a pressure body accommodated in the area of the belt sling, which exerts a compressive force on the extremity when the belt is tensioned. Furthermore, a pressure plate is provided to increase the pressure exerted by the pressure body on the blood vessel located inside the extremity. The essential feature of the technical solution described is that a belt sling is combined with a pressure body and a pressure plate so that the extremity is not tied off around its circumference, but also targeted pressure is exerted on the injured blood vessel.

In addition, an instrument is known from CH 70342 A for ligating injured blood vessels during surgery or after an accident. The medical instrument described has a drum rotatably mounted in a housing, to which straps are attached, the ends of which, protruding from the housing, can be connected by means of a hook element to form a ligation loop. One of the straps is attached to a winding element in the drum in such a way that the loop is pulled together by rotating the winding element. The winding element can be locked in a desired position by means of a locking mechanism, which in turn can be released by pressing a push button. The main disadvantage of the technical solution described is that it has a comparatively complex structure and a large number of different mechanical components and, in addition, it is not possible to move the straps in a controlled manner in order to realize both targeted tightening as well as loosening of the loop.

With regard to the potential dangers of intraoperative bleeding, special demands are placed on the surgeon, particularly during surgery, and especially when performing caesarean sections. More than 30 million caesarean sections are performed worldwide every year. In the period between 2002 and 2015 alone, the number of caesarean sections performed worldwide increased from around 16 million to 29.7 million per year. It can be assumed that the number of caesarean sections performed in the future will continue to rise, since the rate of caesarean sections performed after a first caesarean section has already taken place will also increase as the caesarean section rate rises. Incidentally, in the field of obstetrics, the performance of a further caesarean section for the next pregnancy is always recommended and carried out after two previous caesarean sections. Due to this development, it is also to be expected that the number of peri- and postoperative complications, in particular bleeding complications while performing a procedure, will increase. Especially when opening the uterus and after the child was delivered, there is often a risk of considerable blood loss, wherein the aim is always to keep this as low as possible, especially by closing the opened blood vessel as quickly as possible. However, this is often difficult, since imponderables such as vascular injuries to the uterus, in particular the two uterine arteries or the branches of these arteries, resp., can lead to considerable blood loss within seconds. Atony of the uterus, i.e. poor contraction of the uterine muscles, is likewise a feared event during a caesarean section, which can lead to the uterus having to be removed as an emergency treatment, despite all available support measures.

Based on the solutions known from the state of the art as well as the problems described above, the invention is therefore based on the object of providing a medical instrument with which the blood flow exiting through an injured vessel can be at least considerably reduced, and in the best case stopped, both in a comparatively simple and rapid manner. In this context, the instrument to be specified should preferably also be suitable for a caesarean section, and here in particular for sealing the two uterine arteries and/or their branches, in particular by ring-shaped clamping of the lower uterine segment below the caesarean section. At the same time, handling of the medical instrument should be intuitive for the surgeon, especially without requiring unfamiliar movement patterns. Furthermore, the surgeon should have the option of being able to open an injured blood vessel again in a controlled manner after it has been closed, in particular to check whether the blood flow has stopped in the meantime.

### SUMMARY

A medical instrument (1) for reducing the blood flow through a blood vessel of a human or animal is described, having a band element (2), which is fastened to a handling unit (4) and which has a movable band portion (5), the movement of which makes it possible to change the size and/or shape of a surface area (9), which is enclosed by a loop (6) formed by the band element (2).

The technical solution described is characterized in that at least one fixing means (10) is provided for at least temporarily locking the movable band portion (5), and in that, by actuation of the actuation element (8), the movable band portion (5) can be transferred in a targeted manner from the locked position to a position for increasing or decreasing the surface area (9) enclosed by the loop (6).

### DETAILED DESCRIPTION

Overall, it should be possible to actuate the medical instrument with simple hand movements without this having a detrimental effect on reliable hemostasis. A further object of the invention is to provide a respective medical instrument, which can be manufactured in a comparatively simple manner and preferably has only a few components. In addition, it is desirable, if the medical instrument to be specified is designed in such a way that it can be used several times for one patient, but can otherwise be discarded without difficulty, in particular with regard to the costs involved. Among other things, this should at least create the possibility of using a suitable medical instrument without cleaning or sterilization steps being mandatory.

The object described above is solved using a medical instrument according to claim 1 and a set for manufacturing a medical instrument according to claim 14. Advantageous embodiments of the invention are the subject of the dependent claims and are explained in more detail in the following description with partial reference to FIG. 1.

The invention relates to a medical instrument for largely avoiding intraoperative bleeding, in particular for reducing the blood flow through an injured blood vessel of a human or animal, with a band element fastened to a handling unit with a joining region and having a band portion movable relative to the joining region, which band portion, forming a loop, is guided in an adjusting unit, at least indirectly fastened to the handling unit, with an actuation element in such a way that, due to an actuation of the actuation element, the movable band portion can be moved relative to the joining region and thus a size and/or shape of a surface area enclosed by the loop can be changed. The medical instrument according to the invention is characterized in that at least one fixing means is provided for at least temporarily locking the movable band portion relative to the joining region and in that, by actuation of the actuation element, the movable band portion can be transferred in a targeted manner from the locked position to a position for increasing as well as decreasing the surface area enclosed by the loop. It is therefore of particular importance for the invention that it is possible for a surgeon to either open or close an already existing or formed loop, which may have already been locked in place before, as needed. A controlled movement of the movable band portion in both directions opposite to each other is thus possible, so that the surgeon can, for example, check in the meantime by slightly loosening the loop, whether bleeding has been stopped, and can then, depending on the result of the check, either tighten the loop again or loosen it further.

Essential for the technical solution according to the invention is the suitable combination of an adjusting unit, which has an actuation element, with a band element, which on the one hand can be locked in a desired position by using fixing means, and on the other hand can be moved in opposite directions in a targeted manner by actuating the actuation element. Due to this technical design, the loop formed by the band element, in particular the surface area enclosed by the loop, can be both increased and decreased in a targeted manner. By actuating the actuation element, the movable band portion can be moved in opposite directions within its guide and relative to the joining region fixed to the handling unit in such a controlled manner that a surgeon can easily contract the loop as needed or enlarge it again by the required amount. In this way, the blood flow can not only be stopped in a particularly targeted manner, but it is also possible to release the loop in a targeted manner, so that it is easy to check whether the blood flow has possibly already been stopped in another way or has come to a standstill or whether the loop may need to be tightened again.

In a preferred manner, the instrument according to the invention is preconfigured with a band element, the movable band portion of which is located within the guide of the adjusting unit, in such a way that it is available with a loop already produced in sufficient size. In case of need, the surgeon only needs to grasp the instrument, guide the loop over the blood vessel to be closed and, if necessary, also over neighboring organs or other structures of the patient's body and, in a next step, quickly tighten the loop manually, in particular by pulling at the free end of the band element and/or at an eyelet provided in this area. Subsequently, it is easily possible in a further step to effect a further targeted contraction of the loop by actuating the actuation element, for example in the form of a rotary wheel or a rocker arm. As soon as the blood flow is stopped, the surgeon can now devote his full attention to a necessary treatment or surgical step.

Unintentional opening is reliably prevented by the fixing means provided on the band element in the area of the movable band portion, such as indentations, notches or grooves, which are in operative connection with at least one control element of the adjusting unit, e.g., a gear wheel.

Opening or releasing, resp., the loop formed by the band element of the medical instrument according to the invention can be made possible in a targeted manner by repeated actuation of the actuation element of the adjusting unit, so that a control element moves the movable band portion at least section by section and the loop can be enlarged in a targeted manner by the desired amount and the surgeon can check whether the blood flow has already been stopped.

Advantageously, the band element has an element, such as an eyelet, a ring, a handle, a hook or an insertion part inserted into a preformed groove, in the area of the end opposite the joining region, i.e. at its free end, wherein the element can be easily and safely grasped by a surgeon and thus enables a rapid contraction of the loop formed by the band element and a comparatively rapid reduction of the surface area enclosed by the band element.

In a special embodiment of the invention, it is provided that the fixing means on the band element has latching lugs, latching teeth, grooves, balls, holes, notches and/or hooks formed at least in sections. The band element thus has suitable structures on its movable band portion as fixing means, in which a control and/or latching element of the adjusting unit engages, which is moved by actuation of the actuation element in such a way that the movable band portion of the band element is both movable relative to the joining region and lockable in the desired position. For that, the fixing means of the band element as well as the control and/or latching element of the adjusting unit, which is at least temporarily in operative connection with the actuation element, are designed in such a way that both a controlled movement and a reliable locking of the band element can be ensured. In this context, it is particularly advantageous if the fixing means is operatively connected to the adjusting unit, in particular the actuation element, at least in certain areas.

Preferably, the adjusting unit, in particular the actuation element, has a control, latching and/or locking element, which can be moved in a targeted manner by actuation of the actuation element and which engages, at least in sections, in the fixing means of the band element. This way, the band element can be moved within the guide provided in the adjusting unit relative to the fixed joining region in such a way that this movement can lead both to an increase of the surface area enclosed by the loop formed by the band element and to its decrease.

According to another development of the invention, it is provided that the fixing means has a sliding and/or clamping mechanism or cooperates with such a mechanism. In this context, it is conceivable that clamping is established, at least indirectly, between the band element and the actuation element, provided that the movable band portion is to be locked relative to the fixed joining region. The clamping mechanism preferably has suitable elements, which can be moved by actuation of the actuation element and which can optionally be moved into a position in which the band element is clamped in the guide and thus locked, the band element is displaced by a movement of the clamping mechanism or the clamping mechanism is released, so that the band element can be moved, for example by pulling or pushing the free end.

Furthermore, it is conceivable in a preferred manner that the sliding and/or clamping mechanism is designed in such a way that clamping of the movable band portion of the band element is established by successive actuation of the actuation element and at the same time a movement is executed, so that the band element is displaced by a desired distance in the clamped position, in particular due to a friction existing between the band element and a moving control element of the clamping mechanism.

According to a preferred embodiment, the clamping mechanism is formed by at least one part of the adjusting unit. In that, the clamping mechanism preferably has at least one wheel that can be moved relative to the joining region of the band element. This wheel clamps the band element at least in a first position, at least temporarily, in such a way that it is locked in this position, while the clamped band element is displaced within the guide of the adjusting unit during a movement, in particular a rotary movement, specifically brought about by actuation of the actuation element, so that the surface area enclosed by the loop formed by the band element is selectively increased or decreased in a targeted manner.

It is likewise conceivable that the actuation element has a rotary wheel and/or a rocker arm. When a rocker arm is used, it is preferably designed and mounted in such a way that by tilting the rocker arm, it is possible to either lock the band element or displace and thus move it in a targeted manner.

In a particular further development of the invention, the band element, the handling unit and the adjusting unit are made of a plastic material. In this context, it is conceivable in a particularly advantageous manner that the medical instrument is made of plastic as a disposable article, which can be used several times for one patient during a treatment or a procedure, but is disposed of after a respective treatment.

In a further development of the invention, the handling unit has at least one grip area with a recess, a stop and/or with a surface, which is structured at least in certain areas, in particular a roughened surface. In that, the grip area is designed in such a way that it is ensured at all times that a surgeon can hold the medical instrument securely when grasping the handling unit, in particular that slipping during actuation of the actuation element and/or of the band element is reliably prevented.

Furthermore, it is conceivable that the adjusting unit and the fixing means are designed in such a way that the size and/or shape of the surface area enclosed by the loop can be changed in discrete steps. In this context, discrete steps are understood to mean that individual locking positions are provided, which are usually at equal distances from one another and into which the movable band portion can be brought and in which it can then be locked. In this context, it is conceivable, for example, that the adjusting unit has a wheel with external teeth as a control element and the belt element has corresponding recesses, grooves or ridges at least in the movable belt portion, so that a movement of the movable belt portion takes place depending on the size of the teeth and/or the recesses and their spacing. It is also advantageous if a locking mechanism is provided that reliably prevents unintentional movement of the wheel. Such a locking mechanism has, for example, a locking pin that can be pushed into the external teeth of the wheel, when the desired locking position is reached, and pulled out again to release it or moved out by pressing a push button.

In addition, it is advantageous if the joining region of the band element is designed in such a way that it can be detached from the handling unit together with the band element without tools. Preferably, a hook is provided on the handling unit, into which a loop provided in the joining region of the band element can be hooked or hung. It is likewise possible to slide a pin-like structure, which is preferably attached to the end of the band, into a preformed groove in the handling unit. If necessary, it is therefore possible to release the band element from the handling unit and pull it out of the guide of the adjusting unit in order to replace it with another band element of the same or a different design.

The guide channel of the adjusting unit preferably has a rectangular cross-section, in which the movable band portion of the band element is, on the one hand, securely guided and reliably protected against twisting and kinking and, on the other hand, can be locked as required. In general, other cross-sectional shapes (oval, round or semi-circular, resp., or combinations of these in a band) are also conceivable, which ensure secure guidance of the band element, in particular of the movable band portion.

According to a special further development of the invention, it is conceivable that a surface of the band element facing the blood vessel to be sealed after positioning of the medical instrument has a structured, in particular roughened, surface layer in order to impede slipping on the tissue, for example the uterine tissue, or to avoid it, if possible. Moreover, due to the adjustable tension, only the venous leg of a blood vessel could be closed off, for example to allow intravenous medication to take effect.

Preferably, the medical instrument, in particular the handling unit and/or the band element, is made of polyethylene or propylene, at least in certain areas. A preferred width of the band element is between 0.8 and 1.2 cm, particularly preferred about 1 cm, regardless of the material used. However, this value can also vary depending on the application.

According to a further particular embodiment of the invention, the movable band portion of the band element is at least partially located in a curved and/or bendable guide channel. In this case, the material forming the guide channel, such as a suitable plastic material, is preferably designed to be deformable, so that the guide channel with the band element arranged therein can be deformed as required, so that the operation of the medical instrument and/or its approximation to a blood vessel to be ligated or the placing of the loop around a blood vessel and/or an organ, resp., is simplified.

Furthermore, according to a particular further development, it is conceivable that the guide channel of the medical instrument has an opening at the end, through which the movable band portion of the band element exits at least almost in a straight line and is thus arranged at least approximately parallel to the guide channel in the region of the opening, or that the opening is arranged laterally, in particular on the outer circumference of the guide channel, so that the movable band portion is at least slightly bent in the region of the opening, particularly preferably in front of or through the opening itself with respect to the direction of movement in the guide channel. In that, it is also conceivable that the guide channel itself is designed in a straight or curved or curvable manner.

Moreover, the invention also relates to a set for manufacturing a medical instrument, which is designed according to at least one of the embodiments described above and essentially has a handling unit and at least two band elements. Using such a set, a medical instrument designed according to the invention can be provided so that it can be used quickly on the one hand and, on the other hand, the band element can be replaced, if necessary. In this context, it is generally conceivable that in particular the handling unit, to which the band element is attached with its joining region, is designed as a reusable article made of a metal or plastic and/or that all parts are made of plastic and in this case are designed as so-called disposable or single-use articles.

### BRIEF DESCRIPTION OF THE DRAWINGS

Hereinafter, the invention is explained in more detail, without limiting the general idea of the invention, on the basis of embodiments with reference to FIG. 1. Therein:

FIG. 1: shows a first embodiment of a medical instrument configured according to the invention for avoiding or reducing intraoperative bleeding having an adjusting unit for adjusting the size of a ligation loop in discrete steps,

FIG. 2: shows a second embodiment of a medical instrument configured according to the invention for avoiding or reducing intraoperative bleeding having an adjusting unit for continuous adjustment of the size of a ligation loop, as well as

FIG. 3: shows representations of a third, fourth and fifth embodiment of a medical instrument configured according to the invention for avoiding or reducing intraoperative bleeding having an adjusting unit for adjusting the size of a ligation loop in discrete steps.

### DETAILED DESCRIPTION OF THE PREFERRED EMBODIMENTS

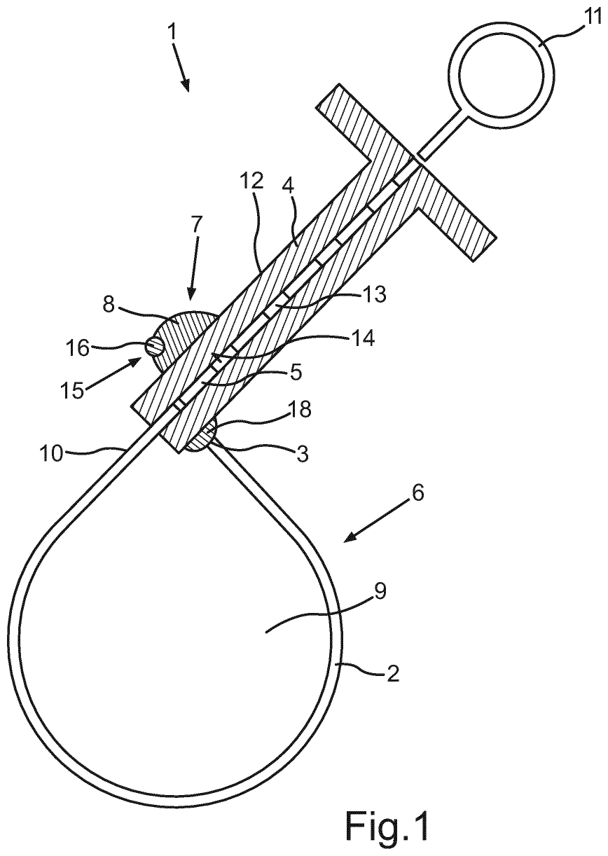

FIG. 1 shows a cross-sectional view of a medical instrument 1 configured according to the invention for reducing or avoiding bleeding during a surgical procedure. In a particularly preferred manner, the medical instrument 1 represented in FIG. 1 can be used during a cesarean section in order to be applied for vascular injuries of the uterus, in particular of the two uterine arteries or their branches, to thus stop the bleeding quickly and efficiently. This is of considerable significance since vascular injuries in this area can lead to high blood loss within seconds. Atony of the uterus, i.e. comparatively poor contraction of the uterine muscles, is likewise a feared event, since heavy bleeding can occur here, too.

Other surgeries, in which the medical instrument 1 represented in FIG. 1 can be used in an advantageous manner are uterine removals, various endoscopic procedures and procedures in intestinal surgery as well as in emergency and military medicine. Not least, the instrument described is also suitable for tensioning surgical regions during plastic surgery.

The medical instrument 1 represented in FIG. 1, in particular its individual components, which are explained in more detail in the following, are made of plastic, so that the illustrated instrument 1 is, in principle, intended for one-time use. This means that the instrument 1 can be used several times for one patient during a procedure, but can then be disposed of without difficulty.

Alternatively, it is conceivable to manufacture in particular the handling unit 4 with the adjusting unit 7 and the actuation element 8 as a multiple-use instrument 1, for example at least partially from a metal, preferably from a precious metal.

The medical instrument 1 designed according to the invention has a handling unit 4 as well as a band element 2, which is fastened to the handling unit 4 in a joining region 3. According to the embodiment represented in FIG. 1, the handling unit 4 has a hook and the band element 2 has a loop in the joining region 3, so that the band element 2 is fastened to the handling unit 4 with its joining region 3 releasable without tools. Furthermore, the band element 2 has a movable band portion 5, which is, at least partially, guided in a guide channel 13 in the handling unit 4 as well as in the adjusting unit 7 in such a way that the band element 2 forms a loop 6 outside the handling unit 4. In that, the movable band portion 5 of the band element 2 can be moved relative to the fixed joining region 3 in such a way that the size and/or shape of a surface area 9 enclosed by the formed loop 6 can be changed as needed, here increased as well as decreased.

At an end opposite the fixed joining region 3, the band element 2 has a gripping element 11, which in the case shown is formed as a ring eyelet. The ring size is designed such that a surgeon can securely grasp it during a surgical procedure and can effect a change in the size of the loop 6 formed by the band element 2, in particular a contraction of the loop 6, in a comparatively fast manner. Furthermore, the handling unit 4 has an adjusting unit 7, via which a targeted movement of the movable band portion 5 of the band element 2 relative to the fixed joining region 3 can be realized. The adjusting unit 7 has an actuation element 8, which according to the embodiment shown is designed in the form of a rotary wheel, and a control element 14, here in the form of a gear wheel, which is moved upon actuation of the rotary wheel, whereby its teeth engage in the fixing means 10 of the band element 2 formed as grooves. Due to the actuation of the rotary wheel, the movable band portion 5 is thus moved into a desired position in a targeted manner, and at the same time the loop 6 or the surface area 9 enclosed by it is decreased or increased to the desired size. In that, it is of particular significance that a movement of the movable band portion 5 in the opposite direction along the guide channel 13 is possible, so that both a targeted enlargement and also reduction of the loop 6 can be undertaken by the surgeon.

If the adjusting unit 7 or the actuation element 8, resp., is not actuated, then the movable band portion 5 of the band element 2 is in a locked position and is reliably held in this position. In addition, it is conceivable that the adjusting unit 7 has a locking mechanism 15, which, upon its activation, transfers the actuation element 5, here the rotary wheel, into a locked position and thus reliably prevents unintentional opening of the loop 6. If in this case the surgeon wishes to carefully open the loop 6, he first actuates the push button 16 provided for deactivation of the locking mechanism 15, to then turn the actuation element 8 designed as a rotary wheel in the desired direction and thus move the movable band portion 5 of the band element 2 according to the representation in FIG. 1 downward bit by bit. By providing individual teeth in the area of the adjusting unit 7 and grooves as fixing means 10 at the band element 2, which are arranged on a side of the band element 2 facing away from the vessel to be ligated, an adjustment of the band element 2 and the change in size of the loop 6 associated therewith can be undertaken in discrete steps.

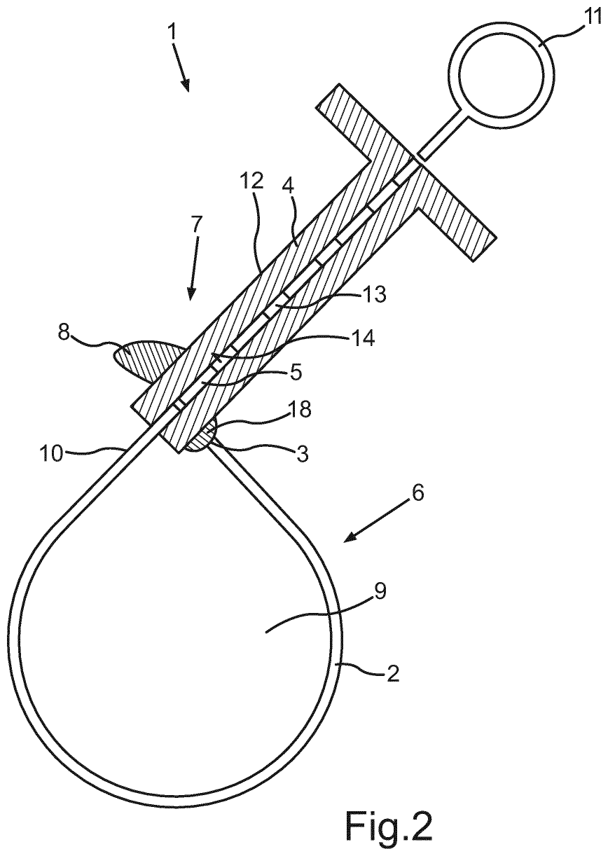

FIG. 2 shows an alternative development of a medical instrument according to the invention, wherein the components not mentioned in the following correspond to components explained in the context of FIG. 1 in regards to their design and function. As a possible alternative it is conceivable that the adjusting unit 7 has a clamping mechanism 17 in operative connection with the actuation element 8, so that the movable band portion 5 of the band element 2 can be clamped at least in certain areas between the guide channel 13 and a clamping element 19. With a suitable movement of the clamping element 19, which is operatively connected with the actuation element 8, the clamped part of the movable band portion 5 is moved relative to the guide channel 13 due to the friction existing between the clamping element 19 and the band portion 5, in particular displaced in the desired direction. If the actuation element 8 with the clamping element 19 is no longer moved, then the movable band portion 5 is reliably locked again within the guide channel 13 and an undesired movement of the band element 2 is reliably prevented. In the solution configured this way, locking of the movable band portion 5 is possible at arbitrary positions, i.e. not only in discrete positions, which have an equal distance from each other. In general, the medical instruments 1 shown in FIGS. 1 and 2 can be used for the same purposes, in particular for the same surgical procedures.

At the beginning of a surgery, in particular before the start of a cesarian section, the band element 2 preferably already is in a position fastened to the handling unit 4, and the movable band portion 5 is positioned within the adjusting unit 7 in such a way that the loop 6 formed by the band element 2 encloses a surface area 9 with an average diameter of about 15 cm. The medical instrument 1 prepared this way can be pulled over the uterus tilted towards the pubic bone, if needed, is then guided up to the desired position behind the source of bleeding and then tightened, wherein the surgeon first grasps the gripping element 13 designed in the form of a ring eyelet and pulls at the band element 2 to thus contract the loop 6. If needed, he can also actuate the actuation element 8, here either the rotary wheel according to FIG. 1 or the actuation element 8 according to FIG. 2, in order to achieve a targeted further contraction of the loop 6. In this manner, the bleeding is initially stopped or at least considerably reduced bleeding is ensured. During this time, life-sustaining and other measures for treating the patient can be performed. As soon as the respective measures or treatment steps are concluded, it is possible for the surgeon to actuate the actuation element 8, for example a rotary wheel or a rocker arm, and thus move the band element 2 and carefully open the loop 6. In this manner, the bleeding tendency prevailing at this time can be checked. If needed, it is possible to contract the loop 6 again.

A medical instrument 1 configured according to the invention thus enables both the closing as well as the controlled opening of a loop 6 provided for avoiding intraoperative bleeding. Thus, at any time after the treatment of the patients, the source of bleeding can be checked by carefully opening of the loop 6 and, if needed, the loop 6 can be contracted again and thus the bleeding stopped again. In this manner, sources of bleeding can be detected and stopped in a secure and uncomplicated manner or manipulations at the uterus, such as administrations of medication into the uterine wall, sutures and the like, can be undertaken. By providing a hook mechanism 18, to which the fixed joining region 3 of the band element 2 can be fastened, it is not absolutely necessary to put a band element 2 over the uterus as a loop, but it is possible to first place it around the vessels to be closed and subsequently fasten it to the handling unit 4. In this case, upon using a respectively designed medical instrument 1, after positioning the first medical instrument 1, it is also possible to position a second one in front of the first one in the direction of the cervix. This way, all potential sources of bleeding proximal and distal to the caesarean section can be temporarily stopped.

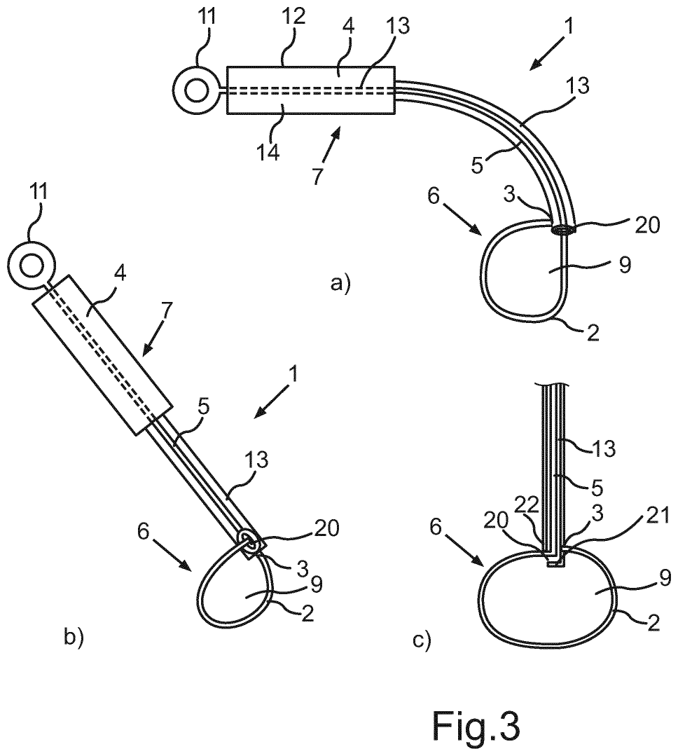

In its representations a), b) and c), FIG. 3 shows a third, fourth and fifth embodiment each of a medical instrument 1 configured according to the invention for avoiding or reducing intraoperative bleeding with an adjusting unit 7 for adjusting the size of a ligation loop 6 in discrete steps. Essential for the medical instruments 1 shown in FIG. 3 is the different design of an opening 20 of the guide channel 13 arranged at the distal end of the instrument 1, through which the movable band portion 5 of the band element 2 exits the guide channel 13. In that, it is essential that the movable band portion 5 exits the opening 20 in a straight line or undergoes an at least partial bend before or while exiting the opening 20.

Furthermore, different designs of the guide channel 13 are shown, which can be configured either straight or curved or even bendable as needed. In that, the individual features stated above can be combined with one another in a different manner as needed. Moreover, the medical instruments 1 shown in FIG. 3 also have the essential features of the invention and can be further developed according to at least one of the embodiments described above.

FIG. 3 a) represents a medical instrument 1 configured according to the invention, in which a loop 6 can be locked in a position and a surface area 9 enclosed by the loop 6 can be increased or decreased in a controlled manner and as needed. The medical instrument 1 has a curved guide channel 13, in which, at least partially, the movable band portion 5 of the band element 2 is located. At the distal end of the guide channel 13, an opening 20 is provided, through which the band element 2 according to this embodiment exits in a straight line, so that the orientation of the band element 2 does not change in the area of the opening 20. The joining region 3 is located at the sheath surface, which limits the outer circumference of the guide channel 13, so that the band element 2 is fastened to the handling unit 4 in this area and fixed relatively thereto. By moving the movable band portion 5, the surface area 9 enclosed by the loop 6 can be either decreased as needed, i.e. contracted, or increased, i.e. released. The curvature of the guide channel enables simplified handling of the instrument 1.

Contrary to the embodiment shown in FIG. 3 a), FIG. 3 b) illustrates a medical instrument 1 designed according to the invention, which has a straight guide channel 13 and an opening 20, through which the movable band portion 5 of the band element 2 laterally exits the guide channel 13 in the area of the circumferential sheath surface. Contrary to the orientation of the band element 2 within the guide channel 13, it undergoes a deflection in the area of the opening 20, here by at least almost 90°. The opening 20 is configured in the form of a through-hole, through which the movable band portion 5 of the band element 2 exits. According to FIG. 3 b), the fixed end of the band element 2 is at the distal end, which limits the guide channel in the longitudinal direction, fastened to the handling unit 4 in the joining region 3 and fixed in the joining region 3 movable relative to the movable band portion 5. The embodiment of a medical instrument 1 shown in FIG. 3 c) differs from the design according to FIG. 3 b) in that the guide channel 13 is bent or folded over, resp., at its distal end, so that the end of the guide channel 13 forms the opening 20, which, however, is inclined by at least almost 90° relative to the main development direction of the guide channel 13. In this case, too, the movable band portion 5 of the band element 2 is thus bent over by at least about 90° in the area of the opening 20.

The opening 20 represented in FIG. 3 c) can be produced in an advantageous manner by making the wall limiting the guide channel 13 in the area of the distal end of the guide channel 13 longer in a first area 21 than in a second area 22 and bending the first area 21 of the guide channel wall as shown in FIG. 3 c).

### LIST OF REFERENCE NUMERALS

1 Medical instrument
2 Band element
3 Joining region
4 Handling unit
5 Movable band portion
6 Loop
7 Adjusting unit
8 Actuation element
9 Surface area enclosed by the loop
10 Fixing means
11 Gripping element
12 Grip area
13 Guide channel
14 Control element
15 Locking mechanism
16 Push button
17 Clamping mechanism
18 Hook mechanism
19 Clamping element
20 Opening
21 First area of the guide channel wall
22 Second area of the guide channel wall

1. A medical instrument (1) for reducing the blood flow through a blood vessel of a human or animal having a band element (2), which is fastened to a handling unit (4) with a joining region (3) and which has a band portion (5) movable relative to the joining region (3), which band portion (5) forms a loop (6) and is guided in an adjusting unit (7), at least indirectly fastened to the handling unit (4), with an actuation element (8) in such a way that, due to an actuation of the actuation element (8), the movable band portion (5) can be moved relative to the joining region (3) and thus a size and/or shape of a surface area (9) enclosed by the loop (6) can be changed, characterized in that at least one fixing means (10) is provided for at least temporarily locking the movable band portion (5) and in that, by actuation of the actuation element (8), the movable band portion (5) can be transferred in a targeted manner from the locked position to a position for increasing as well as decreasing the surface area (9) enclosed by the loop (6).

2. The medical instrument according to claim 1,
characterized in that the fixing means (10) at the band element (2) has latching lugs, latching teeth, grooves, holes, notches and/or hooks formed at least in sections.

3. The medical instrument according to claim 1,
characterized in that the fixing means (10) is in operative connection with the adjusting unit (7), in particular the actuation element (8), at least in certain areas.

4. The medical instrument according to claim 1,
characterized in that the adjusting unit (7) has a clamping mechanism (17).

5. The medical instrument according to claim 4,
characterized in that the clamping mechanism (17) is formed by at least part of the actuation element (8).

6. The medical instrument according to claim 4,
characterized in that the clamping mechanism has at least one wheel movable relative to the joining region (3) of the band element (2).

7. The medical instrument according to claim 1,
characterized in that the actuation element (8) has a rotary wheel and/or a rocker arm.

8. The medical instrument according to claim 1,
characterized in that the band element (2) has a gripping element (11) in the form of an eyelet and/or a hook at an end opposite the fixed joining region (3).

9. The medical instrument according to claim 1,
characterized in that the band element (2), the handling unit (4) and the adjusting unit (7) are manufactured from a plastic material.

10. The medical instrument according to claim 1,
characterized in that the handling unit (4) has at least one grip area (12) with a recess, a stop and/or with a surface structured at least in certain areas.

11. The medical instrument according to claim 1,
characterized in that the adjusting unit (7) and the fixing means (10) are designed such that the size and/or shape of the surface area enclosed by the loop (6) can be changed in discrete steps.

12. The medical instrument according to claim 1,
characterized in that a fastening of the joining region (3) of the band element (2) can be established and released without tools.

13. The medical instrument according to claim 1,
characterized in that the movable band portion (5) is guided in a guide channel (13) with a square cross-section at least in certain areas.

14. The medical instrument according to claim 1,
characterized in that the band element (2) is roughened on a surface, which in a position of use rests against the patient.

15. The medical instrument according to claim 1,
characterized in that the band element (2) has a plastic-coated wire, in particular made of stainless steel or nitinol, at least in sections.

16. The medical instrument according to claim 1,
characterized in that a surface area of the band element (2), which in a position of use rests against the patient, has a width perpendicular to the direction of movement of 0.8 to 1.2 cm, preferably of about 1 cm.

17. The medical instrument according to claim 1,
characterized in that the guide channel (13) has an opening (20) at its distal end, which is formed such that the movable band portion (5) of the band element (2) undergoes a deflection relative to its orientation in the guide channel (13).

18. A set for manufacturing a medical instrument (1) according to claim 1 having a handling unit (4) and at least two band elements (2).
